(19) United States
(12) Patent Application Publication
(10) Pub. No.: US 2024/0279559 A1
(43) Pub. Date: Aug. 22, 2024

(54) HEAT INTEGRATION OF PROCESS COMPRISING A FLUID CATALYST CRACKING REACTOR AND REGENERATOR

(71) Applicant: SHELL USA, INC., Houston, TX (US)

(72) Inventor: Robert Alexander LUDOLPH, Houston, TX (US)

(21) Appl. No.: 18/570,694

(22) PCT Filed: Jul. 7, 2022
(86) PCT No.: PCT/EP2022/068946

§ 371 Date: Dec. 15, 2023

Related U.S. Application Data
(60) Provisional application No. 63/219,926, filed on Jul. 9, 2021.

(51) Int. Cl. C10G 11/18; C07C 5/32

(52) U.S. Cl. CPC C10G 11/182; C07C 5/321; C10G 2300/1092; C10G 2300/4081

(57) ABSTRACT

A heat integration process across two or more industrial processes including a first process in which a hydrocarbon feed is contacted with a regenerated catalyst, passing the hydrocarbon feed and the catalyst admixed therewith through the reactor, thereby converting the hydrocarbon feed and deactivating the catalyst by deposition of carbonaceous deposits thereon, separating the deactivated catalyst from the converted hydrocarbon feed, passing the deactivated catalyst to a regenerator vessel wherein deposits are removed from the deactivated catalyst under exothermic process conditions by means of a regenerating medium, thereby regenerating and heating the catalyst, and passing the regenerated hot catalyst to the upstream section of the reactor, wherein a chemical feedstock for a second process is passed through a heat exchange system in direct contact with the regenerator vessel in order to provide heat to said chemical feedstock and second process.

### FIELD OF THE INVENTION

This invention relates to a process for heat integration across two or more industrial processes for the conversion of hydrocarbons.

### BACKGROUND OF THE INVENTION

The output of a refinery has always shifted in response to market demands for its products. As well as transportation fuel, key commodity chemicals have formed part of a refinery's slate of products for a long time. For example, olefinic and aromatic products have been commercially produced either directly or in downstream processing units linked to refinery feeds as described, for example, in US 20190256786 and US 20200318021.

As energy demands shift, the ability to incorporate the production of chemicals more flexibly within a refinery provides increased value to a refinery owner. Technologies for vaporising or heating hydrocarbon fractions from wide-boiling feedstocks have been investigated as a way to realise this value. Examples of such technology are described in US2016348963, US2015197695, US2010236982, U.S. Pat. No. 4,450,311, GB8625970 and U.S. Pat. No. 4,356,082. It is, however, a challenge to provide the high heat input, often in a staged fashion, required to produce the feedstocks for the chemical production units.

Fluidised bed catalyst units are known in many systems. Within a refinery system, a fluid bed catalytic cracking (FCC) unit generally comprises a riser reactor vessel and a regenerator vessel. In the riser reactor vessel, a hydrocarbon feed is mixed with a catalyst and is cracked at the process temperature. The spent catalyst, containing carbonaceous deposits, is then passed to the regenerator vessel wherein said carbonaceous deposits are removed in an exothermic reaction while contacting the spent catalyst with a regenerating medium such as air.

A number of methods for re-using the heat energy produced in an FCC unit have been described in the art. For example, WO2015001214 discloses a process of heating water in a heat exchange system with a process fluid from an FCC system. Similar methods for producing steam by heat exchange with a catalyst regenerator are also described in US2015197695 and WO2010107541. Combustion of regenerator flue gas to generate electrical power has also been described in the art, for example, in US 2009035193.

Although steam and electrical power are valuable by-products in an industrial process, it is desirable for more efficient systems for conservation of heat energy to be developed. Further, steam is limited in the temperature range that it can be used, and is not suitable, therefore, for the high heat input required to produce the feedstocks for the chemical production units. The further development and integration of refinery and chemical processes to provide a more flexible product slate in an energy efficient manner continues to be a highly desirable aim.

### BRIEF DESCRIPTION OF THE DRAWINGS

FIG. 1 is a schematic drawing of an FCC process suitable as the first process of the present invention.

FIG. 2 is a schematic drawing of a dehydrogenation process suitable as the first process of the present invention.

FIGS. 3 and 4 are schematic drawings of embodiments of the present invention.

### SUMMARY OF THE INVENTION

The present invention provides a heat integration process across two or more industrial processes, said heat integration process comprising:
in a first process, in a fluidised catalyst reactor in which a hydrocarbon feed is contacted with a regenerated catalyst in the upstream section of a reactor, passing the hydrocarbon feed and the catalyst admixed therewith through the downstream section of the reactor, thereby converting the hydrocarbon feed and deactivating the catalyst by deposition of carbonaceous deposits thereon, separating the deactivated catalyst from the converted hydrocarbon feed,
passing the deactivated catalyst to a regenerator vessel wherein deposits are removed from the deactivated catalyst under exothermic process conditions by means of a regenerating medium introduced into the regenerator vessel, thereby regenerating and heating the catalyst, and passing the regenerated hot catalyst to the upstream section of the reactor,
wherein a chemical feedstock for a second process is passed through a heat exchange system in direct contact with the regenerator vessel in order to provide heat to said chemical feedstock and second process.

### DETAILED DESCRIPTION OF THE INVENTION

The present inventors have determined that major efficiencies can be made across a combination of two or more industrial processes by using heat generated in a catalyst regenerator vessel directly to heat up a feed for use in chemical production process. This process has the advantage of avoiding the energy losses associated with the conversion of heat to steam and back again. It also allows the transfer of heat at higher temperatures than allowed for with steam production. The integration of the processes and heat exchange between them increases flexibility of the product slate while reducing energy consumption.

The present invention may be applied in any combination of two or more industrial processes in which a first process involves catalytic conversion of a hydrocarbon feed in a fluid bed riser reactor followed by recovery of the catalyst in an exothermic reaction in a catalyst regenerator reactor; and a second process requires a chemical feedstock at a high temperature.

In a preferable embodiment of the present invention, said first process comprises a fluid catalytic cracking (FCC) process. Thus, in this embodiment, the process comprises the steps of, in a fluidised catalyst bed reactor in which a hydrocarbon feed is contacted with a regenerated catalyst in the upstream riser section of a reactor, passing the hydrocarbon feed and the catalyst admixed therewith through the downstream section of the reactor, thereby cracking the hydrocarbon feed and deactivating the catalyst by deposition of carbonaceous deposits thereon.

An FCC process is used for the conversion of relatively high-boiling hydrocarbons to lighter hydrocarbons boiling in the heating oil or gasoline (or lighter) range. In this process, the hydrocarbon feed is contacted with a particulate cracking catalyst in a fluidised catalyst bed under conditions suitable for the conversion of hydrocarbons. Within the riser reactor, a gaseous fluidising medium transports finely divided catalyst particles through the reactor where they are brought into contact with the hydrocarbon feed as it is injected into the reactor. The stream of fluidised catalyst particles contacted with the hydrocarbon feed are then passed downstream of the hydrocarbon feed injection and the hydrocarbon feed is converted to a cracked product in the presence of the catalyst particles.

At the downstream end of the reactor, the catalyst particles are separated from the cracked product. The separated cracked product passes to a downstream fractionation system. The spent catalyst particles will typically contain a carbonaceous coke deposit. The spent catalyst passes through a stripping section, then to the regenerator vessel where the coke deposited on the spent catalyst during the cracking reaction is burned off, via reaction with oxygen-containing gas, to regenerate the spent catalyst. The resulting regenerated catalyst is then re-used in the reactor.

The oxygen-containing gas comprises one or more oxidants. As used herein, an “oxidant” can refer to any compound or element suitable for oxidizing the coke on the surface of the catalyst. Such oxidants include, but are not limited to air, oxygen enriched air (air having an oxygen concentration greater than 21 vol %), oxygen, oxygen deficient air (air having an oxygen concentration less than 21 vol %), or any combination or mixture thereof.

In other embodiments of the invention, said first process may comprise a different process for hydrocarbon conversion taking place in the reactor and regenerator system. Such processes include, but are not limited to, propane dehydrogenation and isobutane dehydrogenation.

The catalyst regeneration part of the first process in the regenerator is exothermic and produces excess heat. The present invention efficiently uses this heat directly to provide the required heat for a chemical feedstock for use in a second process. The chemical feedstock is passed through a heat exchange system in direct contact with the regenerator vessel. Said heat exchange system suitably comprises a tubular heat exchanger which can be configured to run inside or outside the regenerator vessel.

In one embodiment, the heat exchange system comprises a tubular heat exchanger that passes within the regenerator vessel. Heat exchange systems are known in the art and any suitable system may be used herein. Heat exchangers utilising cooling coils or tubes running through a fluidized catalyst particle bed internal to a regenerator are illustratively shown in U.S. Pat. Nos. 4,009,121, 4,220,622, 4,388,218 and 4,343,634. Such systems allow effective thermal contact with the feedstock passing within the regenerator. However, internal heat exchangers are difficult to retrofit and service.

In a further embodiment, the heat exchange system is in direct contact with the outside of the regenerator vessel. For example, the heat exchange system may form part of a catalyst cooler system which is part of the regenerator vessel.

Catalyst coolers are described, for example, in US20160169506 and U.S. Pat. No. 5,209,287. A catalyst cooler typically comprises a shell and tube-type heat exchanger extending from the wall of the regenerator vessel. Catalyst flows from the regenerator vessel, is cooled by a heat exchange system within the catalyst cooler and is returned to the regenerator vessel. Typically a catalyst cooler also comprises a source of fluidising gas to transport the catalyst particles.

In this embodiment of the invention, the chemical feedstock is passed through the heat exchange system of the catalyst cooler section of the regenerator vessel. This embodiment has the further advantage of being simple to retrofit to existing reactor systems.

The chemical feedstock passed through the heat exchange system is any suitable feedstock for the production of commodity or specialist chemicals in an industrial process. Said commodity or specialist chemicals include, but are not limited to, olefins, such as ethylene, propylene and butylene.

Suitably the chemical feedstock is a feedstock readily available within a refinery installation. For example, the chemical feedstock may include crude oil, crude oil fractions, products derived from natural gas and products from refinery processes.

In one preferred embodiment, the chemical feedstock is a feedstock for an ethylene cracker. As such, the chemical feedstock comprises alkanes such as ethane, propane and higher molecular weight alkanes as well as light fractions of gasoline. Such a feedstock is particularly suitable for the heat integration process of the present invention as the heat requirement for the chemical feedstock for an ethylene cracker is very high and is suitably provided in a staged manner.

In another preferred embodiment, the chemical feedstock is a feedstock for a dehydrogenation process, such as a propane or butane dehydrogenation process.

After the chemical feedstock is passed through the heat exchange system in direct contact with the regenerator vessel in order to provide heat to said chemical feedstock, it is passed directly to a further reactor to allow the second process, i.e. chemical transformation to occur.

These embodiments will be further described below in reference to the illustrative, but non-limiting, Figures.

### DETAILED DESCRIPTION OF THE DRAWINGS

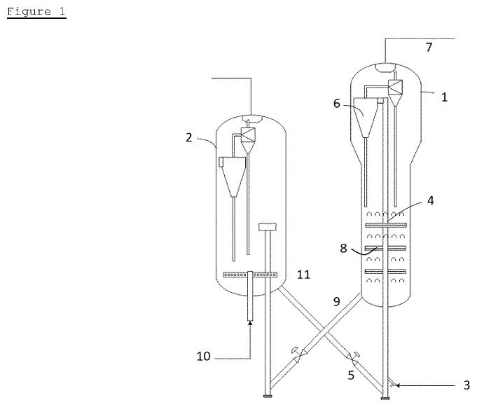

FIG. 1 is a schematic drawing of a fluid catalyst cracking reactor/regenerator system, comprising a reactor 1 and a regenerator 2.

A hydrocarbon feed 3 is injected into an upstream section of the reactor, in this case a riser reactor 4, where it is contacted with the regenerated catalyst supplied via a feed system. The admixed catalyst and hydrocarbon feed pass through the riser reactor, cracking the hydrocarbon and deactivating the catalyst.

In a downstream section 6 of the reactor 1, the deactivated catalyst and cracked product are separated. The spent catalyst passes through a stripping section 8 of the reactor and is then passed through a further feed system 9 to the regenerator vessel 2. Oxygen-containing gas 10 is provided via a gas distribution system 11. Coke, deposited on the spent catalyst during the cracking reaction, is burned off and the regenerated catalyst is passed from the bottom of the regenerator vessel 2, via the feed system 5, for re-use.

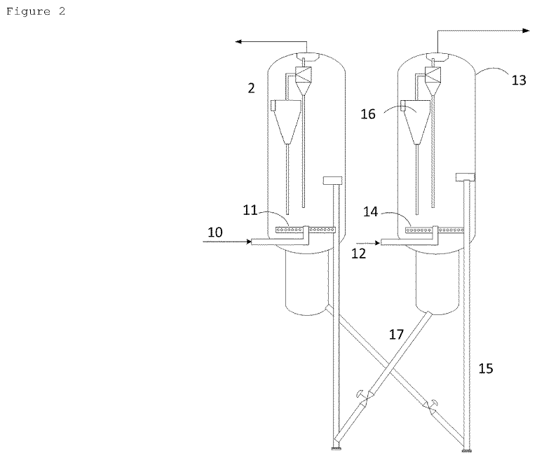

FIG. 2 illustrates a similar reactor system for use in a dehydrogenation reaction.

The dehydrogenation hydrocarbon feed 12 is supplied to an upstream section of a dehydrogenation reactor 13 via a distribution system 14. Catalyst is supplied to the reactor 13 via a feed system 15. The dehydrogenation hydrocarbon feed 12 is contacted with catalyst and is converted, with concurrent deactivation of the catalyst. The deactivated catalyst and hydrocarbon product are separated in a downstream section of the dehydrogenation reactor 16. The deactivated catalyst is passed through a section feed system 17 to a regenerator vessel 2. Oxygen-containing gas 10 is provided via a gas distribution system 11. Coke, deposited on the spent catalyst during the dehydrogenation reaction, is burned off and the regenerated catalyst is passed from the bottom of the regenerator vessel 2, via the feed system 15, for re-use.

In both of the embodiments illustrated in FIGS. 1 and 2, heat is produced in the regenerator vessel 2. In the present invention said heat is used in a heat integration process in that a chemical feedstock is passed through a heat exchange system in direct contact with the regenerator vessel in order to provide heat to said chemical feedstock.

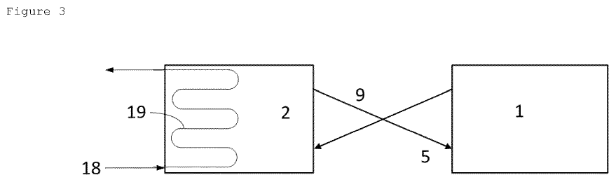

FIG. 3 is a schematic representation of one embodiment of the present invention. FIG. 3 shows a simplified reactor system comprising a reactor 1, a regenerator 2 and feed systems 5, 9 allowing catalyst flow between the two vessels. A chemical feedstock 18 is provided to a heat exchange system 19 which comprises a tubular heat exchanger that passes within the regenerator vessel.

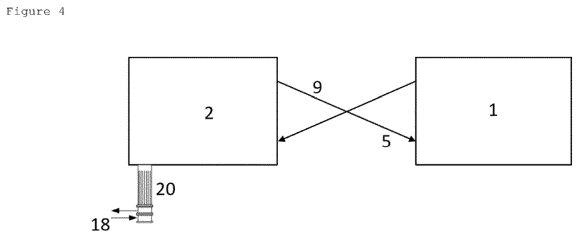

FIG. 4 illustrates the embodiment in which chemical feedstock 18 is provided to a heat exchange system that is in direct contact with the outside of the regenerator vessel. In this embodiment, the heat exchange system forms part of a catalyst cooler system 20 which is part of the regenerator vessel.

1. A heat integration process across two or more industrial processes, said heat integration process comprising:
in a first process, in a fluidised catalyst reactor in which a hydrocarbon feed is contacted with a regenerated catalyst in the upstream section of a reactor, passing the hydrocarbon feed and the catalyst admixed therewith through the reactor, thereby converting the hydrocarbon feed and deactivating the catalyst by deposition of carbonaceous deposits thereon,
separating the deactivated catalyst from the converted hydrocarbon feed,
passing the deactivated catalyst to a regenerator vessel wherein deposits are removed from the deactivated catalyst under exothermic process conditions by means of a regenerating medium introduced into the regenerator vessel, thereby regenerating and heating the catalyst, and passing the regenerated hot catalyst to the upstream section of the reactor,
wherein a chemical feedstock for a second process is passed through a heat exchange system in direct contact with the regenerator vessel in order to provide heat to said chemical feedstock and second process.

2. A heat integration process as claimed in claim 1, wherein the first process comprises a fluid catalytic cracking (FCC) process.

3. A heat integration process as claimed in claim 1, wherein the first process comprises a process selected from propane dehydrogenation and isobutane dehydrogenation.

4. A heat integration process as claimed in claim 1, wherein the heat exchange system comprises a tubular heat exchanger that passes within the regenerator vessel

5. A heat integration process as claimed in claim 1, wherein the heat exchange system is in direct contact with the outside of the regenerator vessel preferably the heat exchange system is part of a catalyst cooler system.

6. A heat integration process as claimed in claim 1, wherein the chemical feedstock is a feedstock for an ethylene cracker.

7. A heat integration process as claimed in claim 1, wherein the chemical feedstock is a feedstock for a dehydrogenation process, selected from a propane or butane dehydrogenation process.
